(19) United States
(12) Patent Application Publication
(10) Pub. No.: US 2016/0081587 A1
(43) Pub. Date: Mar. 24, 2016

(54) Biometric GPS breathalyzer apparatus

(71) Applicant: Ohanes D. Ghazarian, Henderson, NV (US)

(72) Inventor: Ohanes D. Ghazarian, Henderson, NV (US)

(21) Appl. No.: 14/121,595

(22) Filed: Sep. 22, 2014

(51) Int. Cl. A61B 5/08; A61B 5/18; B60K 28/06; A61B 5/00

(52) U.S. Cl. CPC A61B 5/082; A61B 5/0077; A61B 5/18; A61B 5/6893; A61B 5/7465; A61B 5/7405; A61B 5/4833; A61B 5/0013; A61B 5/486; A61B 5/6831; A61B 5/0022; A61B 5/0024; B60K 28/066

(57) ABSTRACT

A Biometric positioning locator breathalyzer apparatus securely attached to a person to be monitored, used to determine user breath alcohol level, wherein when the user bodily speed reach a certain speed threshold. The apparatus initiates a breath test request signal to determine if the person to be monitored is intoxicated. During breath test performance the apparatus video camera captures the video images of the person performing the breath test procedure, and compares with user video image pictures previously stored in the apparatus processor database to ascertain the authenticity of the user is none other than the person to be monitored. If a given breath test reading found to be above certain threshold level, or the user image found to be other than the person to be monitored, or the user fall to perform a breath test, the apparatus will transmit a violation signal along with the user picture and positioning location information to a monitoring station.

This application benefits U.S. provisional application No. 61/960,679 filed on Sep. 23, 2013

### BACKGROUND OF THE INVENTION

There are many different portable alcohol breathalyzers in use by police officers, or by individuals under court mandate program which requires individuals to carry a breathalyzer device to provide sobriety test based on randomly received signal from a monitoring station, or by a breathalyzer generated random signals. These devices utilize a camera to capture the individual picture during a user breath test inhale exhale procedure to identify user. The breathalyzer based on individual given breath sample, it transmits the detected test result to a monitoring station along with user captured photo images for the monitoring station operator to be able to identify the person performing the breath test.

Such prior art systems are useful, wherein when the person to be monitored is not allowed to consume certain amount or no amount of alcohol at any given time. However this sort of teaching cannot know when a person to be monitored starts operating a vehicle, there for it does not automatically prompt the user to give a breath test sample when the person starts operating a moving a vehicle. There for an intoxicated person may take a chance in between random test time period and operate a vehicle without being caught. As such prior art teachings requires the person to be monitored be tested randomly regardless if the person is at work or at a social event, there for it becomes annoying and publicly humiliating for the person to be monitored. Besides prior art teaching is not practical for the monitoring station operator, since it requires the attention of a monitoring personnel to verify the received video images match the authenticity of the user regardless if the user sober or not. Contrary to the present invention the breathalyzer apparatus in response to user bodily movement at certain speed level, the present invention apparatus activates a signaling device for the user to conduct a breath test, to ascertain the individual is not intoxicated while operating a moving vehicle. Further the present invention apparatus generates random signals based on received coordinate speed detection signals or by accelerometer detected speed signals, for the person to be monitored to perform random breath test procedure during the course of operating a moving vehicle to ascertain the individual does not consume alcohol while operating the vehicle. The present invention apparatus provides user facial biometric recognition circuitry, used to automatically authenticate the person to be monitored by the apparatus to eliminate unnecessary calls to a monitoring station for user verification, in particularly when the person to be monitored passes the breath test, thus eliminating the need of monitoring station personnel verification to determine user authenticity.

There for it is the primary objective of the present invention to provide a tamper detection positioning locator breathalyzer apparatus which is securely attached to a person to be monitored, to ascertain the person to be monitored cannot remove the apparatus and operate a vehicle while intoxicated. The present invention positioning locator receiver or an accelerometer is being used to determine the persons physical speed movement, such as when the person to be monitored operating a moving vehicle or is a passenger in a moving vehicle, the apparatus in response to detected persons bodily movement speed, the apparatus initiates a breath test request signal for the person to be monitored to provide a breath test sample into the apparatus breathalyzer to determine person to be monitored is intoxicated while operating a moving vehicle. The present invention apparatus contains a video camera to capture the video images of a person performing the breath test procedure, the apparatus compares the user given breath sample, and user captured video image or picture within the apparatus processor database previously stored user image information to ascertain the authenticity of the user is none other than the person to be monitored. If a given breath test reading found to be above certain alcohol level threshold, or the user captured image found to be other than the person to be monitored, or if the person to be monitored fail to provide a breath test at a set period time, the apparatus will transmit a violation signal along with the user picture, and provide location information to a monitoring station.

It is another objective of the present invention wherein the Breathalyzer apparatus contains an audio circuitry having a microphone and a speaker configured to establish two way voice between a monitoring station and a person to be monitored, accordingly the monitoring station operator can establish verbal communication with the person to be monitored, and the operator be able to give verbal instruction, example the monitoring station operator may ask the user to move around the apparatus to capture proper facial images of the person performing the test, or the monitoring station operator may ask the person to be monitored to move the apparatus to capture certain images of the vehicle interior to determine if the person to be monitored is indeed located in the driver seat of the moving vehicle or seating in the passenger seat during the breath test procedure.

It is another objective of the present invention wherein a person to be monitored is a passenger in a moving vehicle, such that when the breathalyzer apparatus based on detected speed activates the apparatus signaling circuitry for the person to be monitored to provide a breath test sample into the apparatus to determine the persons sobriety condition. The person to be monitored may activate the apparatus video camera and sent a video clip or a picture to the monitoring station indicating the person to be monitored is seating in the passenger compartment of a vehicle, thus eliminating the need to conduct unnecessary breath test procedure in front of other individual(s).

It is another objective of the present invention wherein the breathalyzer apparatus is attached to the person to be monitored with a tamper detection strap having a tamper detection housing, any attempt to tamper with the housing or the strap, or removal of the apparatus, the present invention apparatus transmits a tamper detection signal to a monitoring station.

The present invention breathalyzer apparatus contains a positioning locator receiver, a radio frequency transceiver, a video camera, a breathalyzer and a two way audio circuitry all in a single piece unit. Or preferably the present invention apparatus may be of a two piece devise, a user carry on portable first devise may contain the breathalyzer, a camera, a two way voice circuitry, and an RF transceiver to communicate with the users second device, and the second device is securely attached to the person to be monitored with removal tamper detection circuitry have a positioning locator receiver, and a second radio frequency transceiver used to establish wireless communication with the first device, and a cellular, satellite transceiver module or a radio frequency transceiver used to communicate with a monitoring station. The first and second devises may contain any combination of the components found in the present invention apparatus.

### SUMMARY OF THE INVENTION

A Biometric positioning locator breathalyzer apparatus securely attached to a person to be monitored, used to determine user breath alcohol level, wherein when the user bodily speed reach a certain speed threshold. The apparatus initiates a breath test request signal for the person to be monitored provide a breath test sample, for the apparatus to determine if the person to be monitored is intoxicated. During breath test performance the apparatus video camera captures the video images or a picture of the person performing the breath test procedure, and the apparatus processor compares captured video image or picture with video image pictures previously stored in the apparatus processor database to ascertain the authenticity of the user is none other than the person to be monitored. If a given breath test reading found to be above certain threshold level, or the user image found to be other than the person to be monitored, or the person to be monitored does not perform a breath test at a set time period, the apparatus will transmit a violation signal along with the user picture and positioning location information to a monitoring station.

The present invention Breathalyzer apparatus contains an audio circuitry having a microphone and a speaker designed to establish two way voice between a monitoring station and a person to be monitored, accordingly the monitoring station operator can establish verbal communication with the person to be monitored, and the operator able to give verbal instruction, such as the user to move around the apparatus to capture proper facial images of the person performing the breath test, or the monitoring station operator may ask the person to be monitored to move the apparatus to capture the vehicle interior certain images to determine if the person to be monitored is indeed located in the driver seat of a moving vehicle or seating in the passenger seat compartment during the breath test procedure.

The present invention wherein the breathalyzer apparatus is attached to the person to be monitored with a tamper detection strap having a tamper detection housing, any attempt to tamper with the housing or the strap, or removal of the apparatus, the present invention apparatus transmits a tamper detection signal to a monitoring station.

The present invention breathalyzer apparatus contains a positioning locator receiver, a radio frequency transceiver, a video camera, a breathalyzer and a two way audio circuitry all in a single piece unit. In a preferred embodiment the present invention apparatus may be of a two piece devise, a user carry on portable first devise may contain the breathalyzer, a camera, a two way voice circuitry, and a first RF transceiver to communicate with the users second device, and the second device is securely attached to the person to be monitored with removal tamper detection circuitry have a positioning locator receiver, and a second radio frequency transceiver used to establish wireless communication with the first device, and a cellular, satellite or radio frequency transceiver to communicate with a monitoring station. The first and second devises may contain any combination components used in the present invention apparatus.

### DESCRIPTION OF THE DRAWINGS

FIG. 1. Is the block Diagram

FIG. 2. Illustrates the biometric positioning locator breathalyzer apparatus having a breathalyzer, a camera, a speaker and a microphone, and radio frequency module designed to communicate with a monitoring station, securely attached to a person arm with a tamper detection strap to be monitored.

FIG. 3 Illustrates two piece biometric positioning locator breathalyzer apparatus, first device having a portable breathalyzer, a camera, speaker, a microphone, and a radio frequency communication module. And second device securely attached to the user leg containing a positioning locator, a radio frequency communication module to communicate with a monitoring station, a second radio communication module used to establish communication with the portable breathalyzer, and illustrates a tamper detection strap.

### DETAILED DESCRIPTION OF THE PREFERRED EMBODIMENT

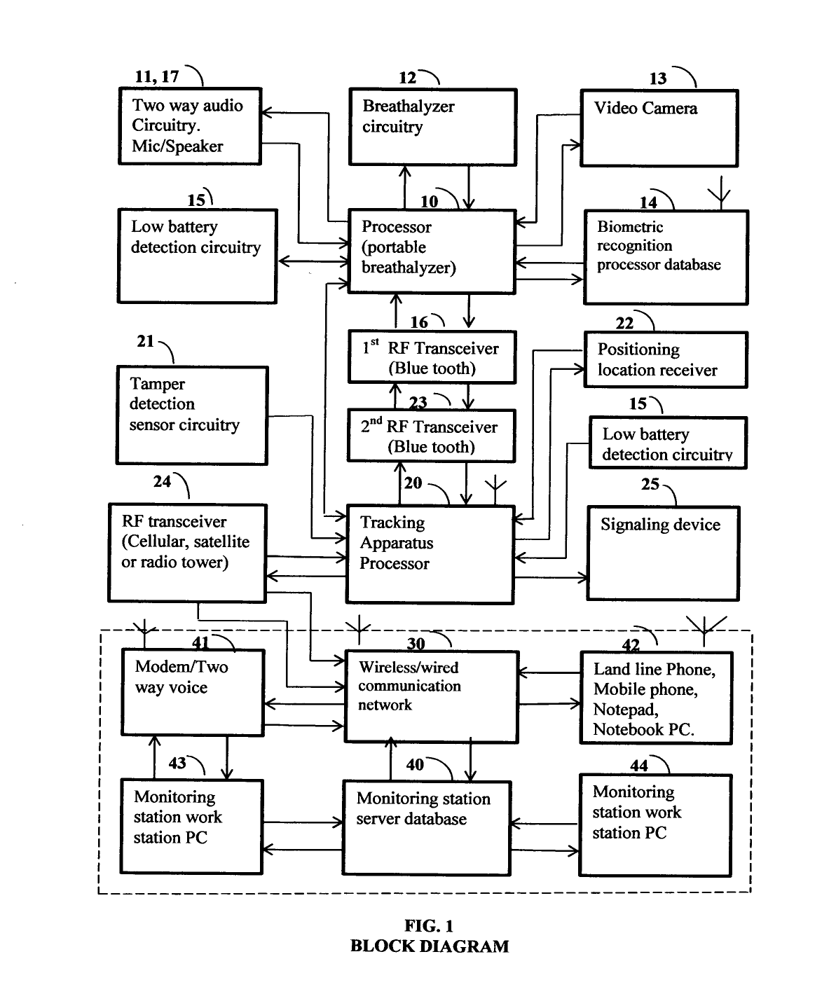

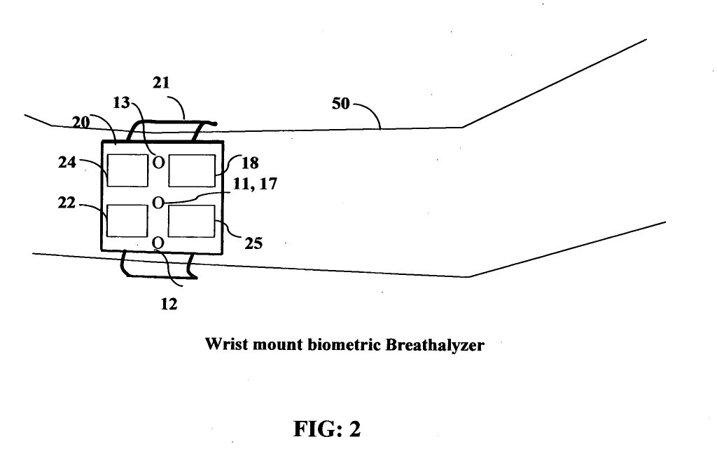

A Biometric positioning locator breathalyzer apparatus FIGS. 1, 2, and 3 securely attached to a person 50 to be monitored, used to determine user breath alcohol level or user breath toxic contents, wherein when a person 50 to be monitored is operating a moving vehicle. The present invention apparatus 20 contains a battery or other power means to supply power to the apparatus 20 electronic circuitry. wherein when the apparatus positing locator 22 detects user 50 bodily speed reach a certain speed threshold, the apparatus 20 signaling circuitry 25 initiates a signal(s) indicative to the person to be monitored 50 at a set time period to give a breath test sample into the apparatus breathalyzer 12, or after the apparatus initiated signal the user may press the apparatus test button 18 for the user perform a breath exhale into the apparatus breathalyzer 12 for the apparatus 20 to determine if the person 50 to be monitored is intoxicated or not. During breath test procedure the apparatus video camera 13 captures the video image or a picture of the person 50 performing the breath test procedure, the apparatus biometric recognition processor 14 compares the captured video image(s) with the person to be monitored video image(s) or picture previously stored in the apparatus processor database 14, to ascertain the authenticity of the user is none other than the person to be monitored 50. If a given breath test reading found to contain above certain threshold level of alcohol, or the person 50 performing the test captured image(s) or picture found to be other than the person 50 to be monitored, or the person to be monitored ignores the apparatus signaling device 25 generated signals for the user 50 to perform a breath test procedure, after a set time period does not perform a breath test procedure, the apparatus processor 20 will transmit a violation signal through the apparatus cellular/Satellite, UHF/VHF radio, or a vehicle blue tooth module to a monitoring station 40, 42, 43, and 44 through a wireless or wireless and wired network containing information to the violation condition, the signal may contains captured video image(s), detected breath test level, apparatus 20 location information.

The apparatus attached to a person 50 to be monitored may utilize an accelerometer to determine the user bodily speed, or may use the apparatus cellular, or radio towers Doppler signal to determine the apparatus is in certain level speed movement and accordingly activate the signaling circuitry 25 for the person 50 to be monitored to perform breath test, the breast test request signals may be generated randomly by the apparatus so long that the apparatus 50 is in certain speed movements.

The present invention apparatus processor 20 may use the apparatus cellular tower signals to determine the apparatus location.

The present invention Breathalyzer apparatus 20 may contains an audio circuitry having a microphone and a speaker 11, 17 designed to establish two way voice between a monitoring station 40, 42, 43, and 44, and the person 50 to be monitored through a wireless, or wireless and wired network 30, accordingly the monitoring station 42, 43, and 44 operator establishes verbal communication with the person 50 to be monitored, the operator may give verbal instructions, such as the user 50 to move around the apparatus to capture proper facial or biometric images of the person 50 to be monitored, or the monitoring station operator 42, 43, and 44 may ask the person 50 to be monitored to move the apparatus to capture the vehicle interior certain images, for the monitoring station operator to determine if the person 50 to be monitored is indeed located in a vehicle driver seat of the moving vehicle or seating in the passenger seat compartment during the given breath test procedure.

It is another objective of the present invention wherein a person 50 to be monitored is located in the passenger compartment of a moving vehicle, such that when the breathalyzer apparatus 20 based on apparatus movement detected speed, activates the apparatus signaling circuitry 25 for the person 50 to be monitored to provide a breath test sample into the apparatus breathalyzer 12 to determine the persons sobriety condition. The person 50 to be monitored at a given preset time presses the test button 18 and activate the apparatus video camera 13, or the apparatus camera may automatically take video image(s) and sent a video clip or a picture to the monitoring station 40, 42, 43, and 44 indicating the person 50 to be monitored is seating in the passenger compartment of a moving vehicle, thus eliminating the need for the person to be monitored 50 to conduct unnecessary breath test procedure in front of other individual(s).

The present invention wherein the breathalyzer apparatus 20 may be attached to the person limb, such as the persons arm 50 with a tamper detection strap 21, the apparatus 20 may contain tamper detection housing, any attempt to tamper with the housing or the strap 21, or removal of the strap 21 or the housing 20 from the person attached too, the present invention apparatus transmits a tamper detection signal to a monitoring station 40, 42, 43, and 44. The tamper detection signal may contain the apparatus location information.

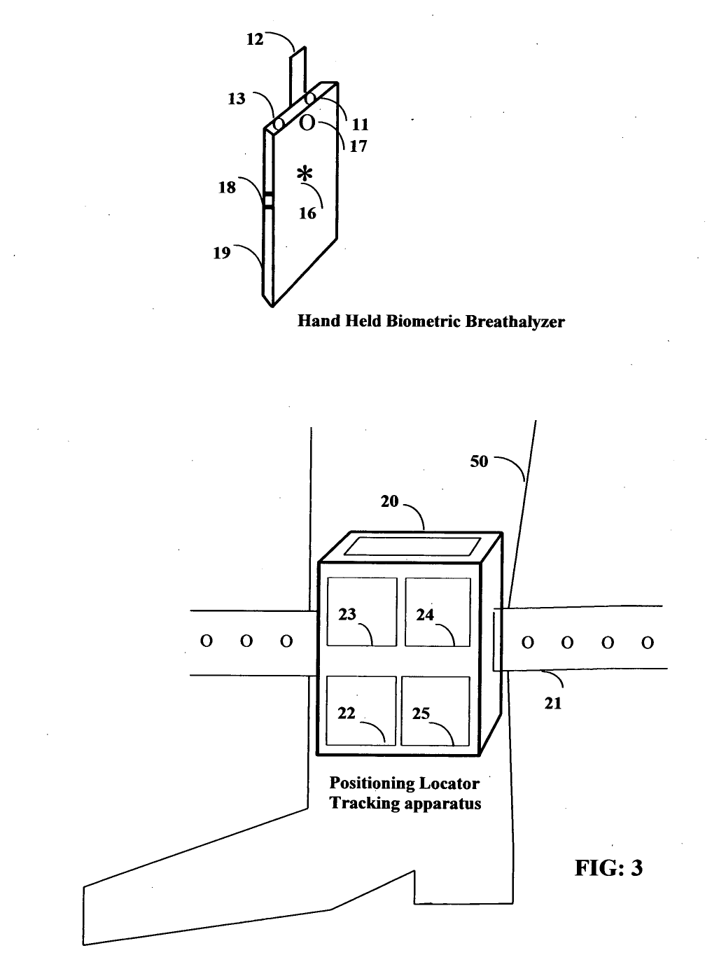

The present invention breathalyzer apparatus 20 electronic circuitry contains a positioning locator receiver 22, a cellular/satellite or radio frequency transceiver 24, a video camera 13, a breathalyzer 12 and a two way audio circuitry 11, 17 all in a single piece unit. In a preferred embodiment of the present invention the apparatus may be of a two piece devise, FIG. 3: a user carry on portable biometric breathalyzer first devise 19, which contains a breathalyzer 12, a camera 13, a two way voice circuitry 11, 17 such as a microphone and an speaker, and a first RF transceiver 16 configured to communicate with the second device 20 RF transceiver 23 wherein when both devices 19, 20 are located within the communication proximity of the other, and the second tracking device 20 is securely attached to the person 50 to be monitored with removal tamper detection circuitry 21, containing a positioning locator receiver 22, and a second radio frequency transceiver 23 used to establish wireless communication with the portable breathalyzer first device 19, and a cellular/satellite or radio frequency transceiver 24 used to communicate with a monitoring station 40, 41, 42, and 44. The first hand held breathalyzer device 19 and second attached tracking devises 20 may contain any combination of the components used in the present invention apparatus.

The present invention apparatus 19, 20 contains a low battery detection circuitry 15 used to activate the signaling circuitry 25 for the user to charge the apparatus battery.

1-10. (canceled)

11- A biometric positioning locator breathalyzer apparatus having an electronic circuitry securely attached to a person to be monitored, used to determine said person's breath toxic level wherein when said person is moving, or when said person is in a moving vehicle above a preset speed level, comprises;
a battery or other power means used to supply power to said apparatus electronic circuitry,
a processor having a memory configured to store a toxic breath setting, and is configured to store said person's video or picture image(s) in said processor memory, and
a positioning locator receiver, used to feed received positioning signals into said processor for said processor to coordinate said person's location, or determine said person's movement, or to determine said person's speed while said person is in a moving vehicle,
a cellular/satellite or radio frequency transceiver used to establish communication with a monitoring station, or other communication devices,
a breathalyzer circuitry used for testing said person's breath toxic level, and feed said detected breath sample into said processor,
a video camera used to capture video or picture image(s) of said person to be monitored, and feed said captured images to said processor, and
a signaling circuitry,
said breathalyzer apparatus processor based on detected speed, activates said signaling circuitry indicative to the person to be monitored at a set time period to give a breath test sample into the apparatus breathalyzer, said video camera captures at least one video image or a picture of said person during performing breath test procedure, said processor compares said given breath sample with previously stored toxic breath setting, and compares captured video image or a picture(s) with said person's video or picture image(s) previously stored in said apparatus processor memory, to ascertain the authenticity of said person performing the breath test is none other than said person to be monitored, if said person given breath test reading found to be above certain toxic threshold level, or said person captured image(s) found to be other than said person to be monitored, or said person to be monitored after a set time period ignores said apparatus signaling device generated signals and does not perform a breath test procedure, said apparatus transmits at least one identifiable signal to a monitoring station.

12- A Biometric positioning locator breathalyzer apparatus as claimed in claim 11, wherein said apparatus processor determines said person's bodily movement speed based cellular/radio frequency transceiver received cell or radio tower Doppler signals, or by received built-in accelerometer signals.

13- A Biometric positioning locator breathalyzer apparatus as claimed in claim 11, wherein said breathalyzer apparatus additionally contains an audio circuitry having a microphone and a speaker configured to establish two way voice communication between a monitoring station and said person to be monitored through a wireless, or wireless and wired network.

14- A Biometric positioning locator breathalyzer apparatus as claimed in claim 11, wherein said person to be monitored is located in the passenger compartment of a moving vehicle, said apparatus upon activating said signaling circuitry, said person to be monitored activates said apparatus video camera and sent a video clip or a picture to the monitoring station indicative that said person to be monitored is located in the passenger compartment of said vehicle, or said apparatus upon activating the signaling circuitry, said apparatus camera may automatically take video image or picture(s) and sent a video clip or a picture(s) to the monitoring station, indicative said person to be monitored is located in the passenger compartment of said vehicle.

15- A Biometric positioning locator breathalyzer apparatus as claimed in claim 11, wherein said breathalyzer apparatus utilizes a tamper detection housing, a tamper detection strap, or both tamper detection housing and a strap, said strap is attached to at least both sides of said apparatus housing to complete said strap tamper detection circuitry, the strap is used to attach said apparatus to said person's limb, any attempt to tamper with said housing, or tamper with said strap, said apparatus transmits at least one identifiable tamper detection signal to a monitoring station.

16- A Biometric positioning locator breathalyzer apparatus as claimed in claim 11, wherein said breathalyzer apparatus signaling circuitry generates random signals for the person to be monitored to perform a breath test procedure as long as the apparatus processor detecting apparatus movement above certain speed level.

17- A Biometric positioning locator breathalyzer apparatus as claimed in claim 11, wherein said breathalyzer apparatus processor determines said apparatus location based on received GPS signals, or by received cellular tower(s) signals.

18- A Biometric positioning locator breathalyzer apparatus as claimed in claim 11, wherein said breathalyzer apparatus processor contains a low battery detection circuitry configured to activate said apparatus signaling circuitry for the person to be monitored to charge said apparatus battery.

19- A Biometric positioning locator breathalyzer apparatus as claimed in claim 11, wherein said breathalyzer apparatus comprises of a two separate devises, a first devise, which contains;
a breathalyzer,
a camera,
a second RF transceiver configured to establish communication with a second device having a secondary RF transceiver, wherein when both devices are located within communication proximity range of the other, said second device having a tamper detection strap used to securely attached said second device to said person's limb to be monitored, said second device contains;
a positioning locator receiver,
a cellular/satellite or radio frequency transceiver used for establishing communication with a monitoring station,
said first device and said second devise components is configured to be utilized in any combination within said first or second device.

20. A Biometric positioning locator breathalyzer apparatus as claimed in claim 19, wherein at list one of said devices utilizes a tamper detection housing, a tamper detection strap, or both tamper detection housing and a strap, said strap is used to attach said device to said person's limb, any attempt to tamper with said housing, or tamper with said strap, said apparatus transmits at least one identifiable tamper detection signal to a monitoring station.

21- A Biometric positioning locator breathalyzer apparatus as claimed in claim 11, wherein said identifiable signal further contains at least one captured video image or a picture of said person performing said breath test, or said apparatus positioning location information, or both said captured video image or picture(s) along with said breathalyzer location information to a monitoring station.

22- A Biometric positioning locator breathalyzer apparatus as claimed in claim 11, wherein said monitoring station operator gives verbal instructions for said person to move around said apparatus to capture proper facial or biometric images of said person to be monitored, or said monitoring station operator give instruction to said person to move said apparatus to capture said vehicle interior certain images, for said monitoring station operator to determine if said person to be monitored is indeed located in driver seat of a moving vehicle or located in the passenger seat compartment during said given breath test procedure.

23- A biometric positioning locator breathalyzer apparatus having an electronic circuitry securely attached to a person to be monitored, used to determine said person's breath toxic level, comprises;
a battery or other power means used to supply power to said apparatus electronic circuitry,
a processor having a memory configured to store a toxic breath setting, and is configured to store said person video or picture image(s) in said processor memory, and
a positioning locator receiver, used to feed received positioning signals into said processor for said processor to coordinate said person location,
a cellular/satellite or radio frequency transceiver used to establish communication with a monitoring station, or other communication devices,
a breathalyzer circuitry used for testing said person breath toxic level, and feed said detected breath sample into said processor,
a video camera used to capture video or picture image(s) of said person to be monitored, and feed said captured images to said processor, and
a signaling circuitry,
said breathalyzer apparatus processor based on monitoring station received signals or based on processor generated random signals activates said signaling circuitry indicative to said person to be monitored at a set time period to give a breath test sample into the apparatus breathalyzer, said video camera captures at least one video image or a picture of said person during performing breath test procedure, said processor compares said given breath sample and captured video image or a picture(s) with said person video or picture images previously stored in said apparatus processor memory, to ascertain the authenticity of said person performing the breath test is none other than said person to be monitored, if said person given breath test reading found to be above certain toxic threshold level, or said person captured image(s) found to be other than said person to be monitored, or said person to be monitored after a set time period ignores said apparatus signaling device generated signals and does not perform a breath test procedure, said apparatus transmits at least one identifiable signal to a monitoring station.

24. A Biometric positioning locator breathalyzer apparatus as claimed in claim 23, wherein said breathalyzer apparatus utilizes a tamper detection housing, a tamper detection strap, or both tamper detection housing and a strap, said strap is attached at least to both sides of said apparatus housing to complete said tamper detection strap circuitry, said strap is used to attach said apparatus to said person limb, any attempt to tamper with said housing, or tamper with said strap, said apparatus transmits at least one identifiable tamper detection signal to a monitoring station.

25- A Biometric positioning locator breathalyzer apparatus as claimed in claim 23, wherein said Biometric breathalyzer apparatus comprises of two separate devises, a first device containing
a breathalyzer,
a camera, and
a second RF transceiver configured to establish communication with a second device having a secondary RF transceiver, wherein when both devices are located within communication proximity range of the other, said second device having a tamper detection strap used to securely attached said second device to said person limb,
said second device contains
a positioning locator receiver,
a cellular/satellite or radio frequency transceiver used for establishing communication with a monitoring station, said first device and said second device components is configured to be utilized in any combination within said first or second device.

26. A Biometric positioning locator breathalyzer apparatus as claimed in claim 24, wherein at list one of said devices utilizes a tamper detection housing, a tamper detection strap, or both tamper detection housing and a strap, said strap is used to attach said apparatus to said person limb, any attempt to tamper with said housing, or tamper with said strap, said apparatus transmits at least one identifiable tamper detection signal to a monitoring station.
